(19) United States
(12) Patent Application Publication
(10) Pub. No.: US 2003/0108901 A1
(43) Pub. Date: Jun. 12, 2003

(54) Serological test for ISAV in fish

(76) Inventors: Frederick S.B. Kibenge, Charlottetown (CA); Molly Kibenge, Charlottetown (CA)

Correspondence Address: SMART & BIGGAR, P.O. BOX 2999, STATION D, 55 METCALFE STREET, SUITE 900, OTTAWA, ON, K1P5Y6, CA

(21) Appl. No.: 10/196,287

(22) Filed: Jul. 17, 2002

Related U.S. Application Data
(60) Provisional application No. 60/305,607, filed on Jul. 17, 2001.

(51) Int. Cl. C12Q 1/70; C12Q 1/68; G01N 33/53; G01N 33/537; G01N 33/543

(52) U.S. Cl. 435/6; 435/5; 435/7.95; 435/7.94

(57) ABSTRACT

There is disclosed immune assays and kits for detecting antibodies to infectious salmon anaemia virus (ISAV). The method comprises contacting the ISAV or ISAV antigen with an antibody-containing sample from fish, and detecting specific binding between the antibody and the ISAV or ISAV antigen.

### REFERENCE TO RELATED APPLICATIONS

This application claims the benefit of U.S. Provisional Application No. 60/305,607, filed Jul. 17, 2001, the content of which is herein incorporated by reference.

### FIELD OF THE INVENTION

The present invention is related to the field of serology. In particular, it is directed to methods for determining the presence of fish viruses or antibodies to fish viruses.

### BACKGROUND OF THE INVENTION

Infectious salmon anaemia (ISA) virus (ISAV), a new orthomyxovirus-like virus, is an important viral pathogen of fish. It causes ISA in marine-farmed Atlantic salmon (Salmo salar L.) characterized grossly by exophthalmia, pale gills, ascites (Thorud & Djupvik 1988), and microscopically by haemorrhagic liver necrosis (Evensen, Thorud & Olsen 1991) and renal interstitial haemorrhage and tubular nephrosis (Mullins, Groman & Wadwoska 1998). The disease was first identified in Norway in 1984 (Thorud & Djupvik 1988) and subsequently in New Brunswick, Canada, in 1996 (Byrne, MacPhee, Ostland, Johnson & Ferguson 1998; Mullins et al. 1998; Lovely, Dannevig, Falk, Hutchin, MacKinnon, Melville, Rimstad & Griffiths 1999), in Scotland, UK, in 1998 (Rodger & Richards 1998), in Nova Scotia, Canada (Ritchie, Cook, Melville, Simard, Cusack & Griffiths 2001) and in Faroe Islands, Denmark, in 2000 (Anon. 2000a), and most recently in Maine, USA (Anon. 2000b). The virus has also recently been detected in diseased Coho salmon in Chile (Kibenge, Gárate, Johnson, Arriagada, Kibenge & Wadowska 2001). Rainbow trout, brown trout, wild Atlantic salmon (Nylund, Alexandersen, Løvik & Jakobsen 1994; Nylund, Alexandersen, Rolland & Jakobsen 1995; Nylund, Kvenseth, Krossøy & Hodneland 1997), herring, and eel have been shown to be asymptomatic carriers of the virus. The virus was identified as being an orthomyxovirus-like virus because it was shown to have ultrastructural and molecular similarities to influenza viruses (Mjaaland, Rimstad, Falk & Dannevig 1997; Falk, Namork, Rimstad, Mjaaland & Dannevig 1997).

Laboratory diagnostic methods used to confirm a diagnosis of ISA are still subject to debate by the regulatory authorities in countries where ISA is known to occur, because of the stringent disease control measures in place. Thus, currently, laboratory diagnosis of ISA includes the isolation of ISAV in SHK-1 cells (Dannevig, Falk & Namork 1995) and/or CHSE-214 cells (Bouchard, Keleher, Opitz, Blake, Edwards & Nicholson 1999; Kibenge, Lyaku, Rainnie & Hammell 2000a) and electron microscopic examination of a positive isolate, and the use of reverse transcriptase-polymerase chain reaction (RT-PCR) (Mjaaland et al., 1997; Kibenge, Whyte, Hammell, Rainnie, Kibenge & Martin 2000b; Rimstad, Falk, Mikalsen & Teig 1999) and indirect fluorescent antibody test (IFAT) on positive virus isolates or on tissue samples from suspected fish (Falk, Namork & Dannevig 1998). Thus no serological test is currently used in diagnosis of ISA, and while vaccination is permitted in areas like the Bay of Fundy, New Brunswick, detection of ISAV infection is sufficient to trigger a decision to depopulate the affected farm.

In contrast to diagnostic tests for infectious diseases of mammals, birds, and bees (Anon. 2000c), confirmation of exposure to a disease agent by detection of antibody to the agent is rarely, if ever, used in aquatic animal disease diagnosis. Present limitations are due not only to lack of experience but also to discrete features of the humoral immune response in fish (Denzin & Staak 2000) such as a predominant IgM subtype (Manning 1994), absence of switch in B-lymphocytes to high affinity binding antibodies (Wilson & Warr 1992), absence of affinity maturation (Wilson, Hsu, Marcus, Courtet, Du Pasquier & Steinberg 1992) and lack of diversity in the antibodies produced (Du Pasquier 1982).

Fish serology (for example virus neutralization and antibody ELISA) could be of importance for detecting carrier status among fish stocks for various viral diseases but is not yet validated, and no antibody detection test is yet approved as an official procedure for routine diagnostic purposes. There remains a need for a serological test for detecting ISAV in fish.

### SUMMARY OF THE INVENTION

The present invention is directed to methods and kits for detecting fish antibodies to infectious salmon anaemia virus (ISAV).

Therefore, there is provided a method for detecting the presence or absence of antibodies to infectious salmon anaemia virus (ISAV), the method comprising the steps of: (a) providing an ISAV or an ISAV antigen; and (b) contacting the ISAV or ISAV antigen from step (a) with an antibody-containing sample from fish, for a time and under conditions sufficient for the antibody to form a complex with the ISAV or ISAV antigen; wherein specific binding between the antibody and the ISAV or ISAV antigen indicates that the antibody is specific to ISAV.

In one embodiment, the method above is used for testing whether a fish is or has been exposed to ISAV, wherein the antibody-containing sample is from the fish being tested, and wherein specific binding between the antibody and the ISAV or ISAV antigen indicates that the fish is or has been exposed to ISAV.

In another embodiment, the fish has not been vaccinated, and specific binding between the antibody and the ISAV or ISAV antigen indicates that the fish has been or is infected with ISAV.

The methods described above may further comprise the step of detecting the complex between the antibody and the ISAV or ISAV antigen. In one embodiment, the ISAV or ISAV antigen is immobilized. In another embodiment, specific binding between the antibody and the ISAV or ISAV antigen is detected by a labeled anti-fish antibody which binds to the complex between the antibody and the ISAV or ISAV antigen.

In another embodiment, specific binding between the antibody and the ISAV or ISAV antigen is detected by contacting an immobilized anti-fish antibody with the complex between the antibody and the ISAV or ISAV antigen, wherein the ISAV or ISAV antigen is labeled.

In one embodiment, the antibody-containing sample from fish has been heat-treated prior to contacting the ISAV or ISAV antigen. The antibody-containing sample from fish may be a fish extract, or fish serum.

The present invention also encompasses a kit for detecting the presence or absence of antibodies to ISAV in an antibody-containing sample from a fish. The kit comprises: (a) an ISAV antigen; and (b) means for detecting a complex between the ISAV antigen and the antibody from the fish.

In one embodiment of the kit, the ISAV antigen is immobilized. In another embodiment, the means for detecting the complex is a labeled anti-fish antibody. The means for detecting the complex may also be an immobilized anti-fish antibody, where the ISAV antigen is labeled.

The present invention also describes a method for testing whether a fish is or has been exposed to ISAV. The method comprises the steps of: (a) providing an ISAV or an ISAV antigen; and (b) contacting the ISAV or ISAV antigen from step (a) with an antibody-containing sample from the fish being, for a time and under conditions sufficient for the antibody to form a complex with the ISAV or ISAV antigen; (c) detecting the complex between the antibody and the ISAV or ISAV antigen; wherein specific binding between the antibody and the ISAV or ISAV antigen indicates that the fish is or has been exposed to ISAV.

In one embodiment, the ISAV or ISAV antigen is immobilized on a solid support and the complex between the antibody and the ISAV or ISAV antigen is detected by detecting the antibody bound to the solid support.

In another embodiment, the complex between the antibody and the ISAV or ISAV antigen is detected by contacting a labeled anti-fish antibody with the complex and detecting the labeled anti-fish antibody bound to the complex.

In another embodiment where the ISAV or ISAV antigen is labeled, the complex between the antibody and the ISAV or ISAV antigen is detected by contacting the complex with an anti-fish antibody immobilized on a solid support and detecting the labeled ISAV or ISAV antigen bound to the solid support.

### BRIEF DESCRIPITION OF THE DRAWINGS

FIG. 1 depicts graphs for determining the optimum coating concentration of virus antigen for use in indirect ELISA for fish antibodies to ISAV. (A) ISAV isolate RPC/NB 990-02-4. (B) ISAV isolate RPC/NB-970877-2. ●=2.5 μg/ml, ▪=7.5 μg/ml, ▾=10 μg/ml.

FIG. 2 depicts graphs indicating corrected mean OD reading in the indirect ELISA of known positive and negative fish serum samples after (A) 10 minutes incuabtion, (B) 60 minutes incubation, (C) 120 minutes incubation, and (D) 240 minutes incubation with the enzyme substrate for Alkaline Phosphatase. ●=strong positive fish serum from field, ▪=weak positive fish serum collected at 6 weeks post inoculation in experimental ISAV infection, ▾=negative fish serum.

FIG. 3 depicts graphs comparing mean OD in ISAV positive and negative fish serum with or without ISA rabbit antiserum reacted against viral and cellular antigens (A) ISAV positive fish serum against ISAV antigen; (B) ISAV positive fish serum against SHK-1 antigen; (C) ISAV negative fish serum against ISAV antigen; (D) ISAV negative fish serum against SHK-1 antigen. ●=OD with no rabbit ISAV antiserum, ▪=OD with rabbit ISAV antiserum. The rabbit ISAV antiserum (1:200) was added to each well.

### DETAILED DESCRIPTION OF THE PREFERRED EMBODIMENTS

The present method is generally based on an immunoassay, such as ELISA (enzyme-linked immunosorbent assay), for detection of fish antibodies to ISAV.

The term “immunoassay” as used herein refers to an analytical method which uses the ability of an antibody to bind a particular antigen as the means for determining the presence of the antigen or antibody. An antibody-capture immunoassay is an assay that provides an antigen which is used to detect antibodies against a particular pathogen in a biological sample of a test subject. In general, the antigen is immobilized on a support and is capable of binding an antibody in a biological sample. The antibody is provided by the biological sample. In a variation of the antibody-capture assay the antigen is mixed with the antibody in the biological sample and the antigen-antibody complex thus formed is captured by a second antibody against the antigen or antibody or both in the antigen-antibody complex which is immobilized on a support. Alternatively, the formation of the antigen-antibody complex is measured in solution.

It is contemplated that a range of immunoassay formats be encompassed by this definition, including but not limited to direct immunoassays, indirect immunoassays, and “sandwich” immunoassays. A particularly preferred format is a sandwich enzyme-linked immunosorbent assay (ELISA). However, it is not intended that the present invention be limited to this format. It is contemplated that other formats, including radioimmunoassays (RIA), immunofluorescent assays (IFA), and other assay formats, including, but not limited to, variations on the ELISA method will be useful in the method of the present invention. Thus, other antigen-antibody reaction formats may be used in the present invention, including but not limited to “flocculation” (ie., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen); “complement fixation” (ie., the use of complement in an antibody-antigen reaction method), and other methods commonly used in serology, immunology, immunocytochemistry, histochemistry, and related fields.

Detection of an antibody-antigen complex can be performed by several methods. The mobile antigen may be prepared with a label such as biotin, an enzyme, a fluorescent marker, or radioactivity, and may be detected directly using this label. Alternatively, a labelled “secondary antibody” or “reporter antibody” which recognizes the primary antibody may be added, forming a complex comprised of antigen-antibody-antibody. Again, appropriate reporter reagents are then added to detect the labelled antibody. Any number of additional antibodies may be added as desired. These antibodies may also be labelled with a marker, including, but not limited to an enzyme, fluorescent marker, or radioactivity. Either the antigen or the antibody (primary or secondary) may be immobilized on a solid support, but the labeled component cannot be immobilized because the detectable signal is precluded from being a measure of binding.

As used herein, the term “reporter reagent” is used in reference to compounds which are capable of detecting the presence of antibody bound to antigen. For example, a reporter reagent may be a calorimetric substance which is attached to an enzymatic substrate. Upon binding of antibody and antigen, the enzyme acts on its substrate and causes the production of a color. Other reporter reagents include, but are not limited to fluorogenic and radioactive compounds or molecules. This definition also encompasses the use of biotin and avidin-based compounds (e.g., including compounds but not limited to neutravidin and streptavidin) as part of the detection system. In one embodiment of the present invention, biotinylated antibodies may be used in the present invention in conjunction with avidin-coated solid support.

As used herein, the term “solid support” is used in reference to any solid material to which reagents such as antibodies, antigens, and other compounds may be attached. For example, in the ELISA method, the wells of microtiter plates often provide solid supports. Other examples of solid supports include microscope slides, coverslips, beads, particles, cell culture flasks, as well as many other items.

A kit for detecting antibodies to ISAV generally comprises, in an amount sufficient for at least one assay, an ISAV antigen and means for detecting a complex between the ISAV antigen and the antibody from the fish, as packaged immunochemical reagents. Instructions for use of a packaged immunochemical reagent are also typically included.

As used herein, the term “packaged” can refer to the use of a solid matrix or material such as glass, plastic, paper, fiber, foil and the like capable of holding within fixed limits an antibody of this invention. Thus, for example, a package can be a glass vial used to contain milligram quantities of a contemplated antigen or it can be a microtiter plate well to which microgram quantities of a contemplated antigen has been operatively affixed. Alternatively, a package could include antigen-coated microparticles entrapped within a porous membrane or embedded in a test strip or dipstick, etc. Alternatively, the antigen can be directly coated onto a membrane, test strip or dipstick, etc. which contacts the sample fluid. Many other possibilities exist and will be readily recognized by those skilled in this art.

Instructions for use typically include a tangible expression describing the reagent concentration or at least one assay method parameter such as the relative amounts of reagent and sample to be admixed, maintenance time periods for reagent/sample admixtures, temperature, buffer conditions and the like.

In preferred embodiments, a kit of the present invention further includes a label or indicating means capable of signaling the formation of a complex between the ISAV antigen and the fish antibody of the present invention.

As used herein, the terms “label” and means for detecting the antibody-antigen complex (“indicating means”) refer to molecules that are either directly or indirectly involved in the production of a detectable signal to indicate the presence of a complex. Any label or indicating means can be linked to or incorporated in an expressed protein, peptide, or antibody molecule that is part of the present invention, or used separately, and those atoms or molecules can be used alone or in conjunction with additional reagents. Such labels are themselves well known in clinical diagnostic chemistry.

The labeling means can be a fluorescent labeling agent that chemically binds to antibodies or antigens without denaturing them to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-natpthalenesulfonyl chloride (DANSC), tetramethylrhodamine isothiocyanate (TRITC), lissamine, rhodamine 8200 sulphonyl chloride (RB 200 SC) and the like.

In preferred embodiments, the indicating group is an enzyme, such as horseradish peroxidase (HRP), glucose oxidase, or the like. In such cases where the principle indicating group is an enzyme such as HRP or glucose oxidase, additional reagents are required to indicate that a receptor-ligand complex (immunoreactant) has formed. Such additional reagents for HRP include hydrogen peroxide and an oxidation dye precursor such as diaminobenzidine. An additional reagent useful with glucose oxidase is 2,2,-azino-di-(3-ethyl-benzthiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Particularly preferred is ¹²⁵I. Another group of useful labeling means are those elements such as ¹¹C, ¹⁸F, 15O and ¹³N which themselves emit positrons. Also useful is a beta emitter, such as ¹¹¹indium or ³H.

The linking of labels, i.e., labeling of peptides and proteins is well known in the art. For instance, monoclonal antibodies produced by a hybridoma can be labeled by metabolic incorporation of radioisotope-containing amino acids provided as a component in the culture medium. The techniques of protein conjugation or coupling through activated functional groups are particularly applicable.

The methods and kits of this invention can also include, preferably as a separate package, a “specific binding agent,” which is capable of selectively binding an antibody or antigen of this invention or a complex containing such a species, but is not itself antigen or antibody of this invention. Exemplary specific binding agents are second antibody molecules, e.g. anti-fish antibodies, complement proteins or fragments thereof, S. aureus protein A, and the like. Preferably the specific binding agent binds the antibody or antigen when it is present as part of a complex.

In preferred embodiments, the specific binding agent is labeled. However, when the method or kit includes a specific binding agent that is not labeled, the agent is typically used as an amplifying means or reagent to amplify the signal. In these embodiments, the labeled specific binding agent is capable of specifically binding the amplifying means when the amplifying means is bound to a complex.

The kits of the present invention can be used in an “ELISA” format to detect the quantity of anti-ISAV antibody in a fish fluid sample or extract. “ELISA” refers to an enzyme linked immunosorbent assay such as those discussed above, which employ an antibody or antigen bound to a solid phase and an enzyme-antigen or enzyme-antibody conjugate to detect and quantify the amount of an antigen present in a sample.

In a number of embodiments, an ISAV antigen can be affixed to a solid matrix to form a solid support. A reagent is typically affixed to a solid matrix by adsorption from an aqueous medium although other modes of affixation applicable to proteins and peptides well known to those skilled in the art, can be used.

Useful solid matrices are also well known in the art. Such materials are water insoluble and include the crosslinked dextran available under the trademark SEPHADEX from Pharmacia Fine Chemicals (Piscataway, N.J.); agarose; polystyrene beads about 1 micron to about 5 millimeters in diameter polyvinyl chloride, polystyrene, crosslinked polyacrylamide, nitrocellulose- or nylon-based webs such as sheets, strips or paddles; or tubes, plates or the wells of a microtiter plate such as those made from polystyrene or polyvinylchloride.

The immunoreagents of any diagnostic system described herein can be provided in solution, as a liquid dispersion or as a substantially dry powder, e.g., in lyophilized form. Where the indicating means is an enzyme, the enzyme's substrate can also be provided in a separate package. A solid support such as the above-described microtiter plate and one or more buffers can also be included as separately packaged elements in the diagnostic assay systems of this invention.

Preferably, the ISAV antigen is coated or adsorbed on to the surface of a substrate. A sample of interest is contacted with the ISAV antigen and any antibodies to the antigen which may be present in the sample bind to the antigen. Anti-fish antibodies are contacted with the antigen/sample and bind to the antibodies that are bound to the ISAV antigen.

The anti-fish antibodies may be labelled with a label. The label is any entity that is capable of being conjugated or bound to the anti-fish antibody and that is capable of being detected by an analytical technique. The label may be conjugated or bound to the anti-fish antibody prior to or after contacting the anti-fish antibody with the antigen/sample.

Detection of the label is an indication that the fish antibody is present in the sample. If the label cannot be detected, then this is an indication that the fish antibody is not in the sample. Since the presence, in the sample, of the fish antibody to ISAV antigen is presumed to arise from ISAV infection or vaccination of the original fish, the presence or absence of the fish antibody is an indication of whether the fish is, or has been, exposed to the ISA virus.

In one embodiment, the label may be a chemical moiety capable of being detected by an analytical technique, the chemical moiety being conjugated to the anti-fish antibody. In this embodiment, the chemical moiety is generally conjugated to the anti-fish antibody before the anti-fish antibody is contacted with the antigen/sample.

In another embodiment the label may be another antibody or collection of other antibodies having conjugated thereto a chemical moiety that is capable of being detected by an analytical technique. In this embodiment, the other antibody or collection of other antibodies having the chemical moiety conjugated thereto is generally bound to the anti-fish antibody after the anti-fish antibody is contacted with the antigen/sample.

In a preferred embodiment, the method further comprises a washing step between steps (b) and (c), and more preferably between each of the steps of the method. Washing is preferably accomplished using a washing solution comprising a buffer, such as phosphate buffered saline solution containing about 1% normal serum from the animal species in which the antibody to which the chemical moiety is conjugated was prepared. An emulsifier may also be present in the washing solution.

ISAV antigens are anything that is capable of raising antibodies to the infectious salmon anaemia virus. Such antigens may be purified or unpurified ISA viruses, proteins or protein fragments thereof, or analogs or homologs of ISAV proteins or protein fragments. Such antigens may also be recombinant ISA viruses, or proteins or protein fragments thereof. Particularly preferred antigens are purified ISA viruses, recombinant ISA viruses, or proteins thereof.

The sample is anything that potentially may contain antibodies to ISAV. Typically, some sample preparation is required and may be accomplished by methods known in the art (for example: Beard, C. W. Serologic procedures. In: Isolation and identification of avian pathogens. 2 nd edn. The American Association of Avian Pathologists. pp 40, and, Dixon P. F., Hattenberger-Baudouy A. -M. & Way K. (1994) Detection of carp antibodies to spring viraemia of carp virus by a competitive immunoassay. Diseases of Aquatic Organisms 19, 181-186; the relevant portions of these documents being hereby incorporated by reference). The sample is preferably taken from a biological source, in particular from a fish. More preferably, the sample is fish body fluid, or an extract or serum from a fish. Salmon, such as Atlantic and Coho salmon, are particular examples of the type of fish for which the present method or assay is useful.

Anti-fish antibodies may be any antibodies that bind to the fish antibodies in the sample to be tested. Examples of anti-fish antibodies are mouse monoclonal anti-fish antibodies and rabbit mono-specific polyclonal anti-fish antibodies. Examples of mouse monoclonal anti-fish antibodies are mouse monoclonal antibody IPA3D1 to Atlantic salmon immunoglobulin and mouse monoclonal antibody IPA2C7 to Rainbow trout immunoglobulin.

When another antibody or collection of other antibodies is used in the label, at least one of these other antibodies binds to the anti-fish antibodies. For example, antibodies that bind to the anti-fish antibody may be raised in a variety of animal species, particularly mammals such as goat and pig species. An example of such an antibody is a goat anti-mouse IgG-1 antibody.

A variety of chemical moieties capable of being detected by an analytical technique and capable of being conjugated to an antibody may be used in the label. For instance, the chemical moiety may be capable of fluorescence or radioactivity (see Fuller S. A., Evelegh M. J. & Hurrell J. G. R. (2000) Conjugates of Enzymes to Antibodies. In: Current Protocols in Molecular Biology. (Eds. Ausubel F. M., Brent R., Kingston R. E., Moore D. D., Seidman J. G., Smith J. A. & Struhl K.) John Wiley & Sons, Inc. Vol. 2, pp. 11.1.1-11.1.7, and, Sambrook J., Fritsch E. F. & Maniatis T. (1989) Molecular cloning. A laboratory manual. 2 nd edn. Cold Spring Harbor Laboratory Press. Cold Spring Harbor, N.Y., the disclosure of both being hereby incorporated by reference). An example of chemical moiety useful in this invention is alkaline phosphatase. Further treatment may be required before the analytical technique is used to detect the label depending on the particular technique or chemical moiety being used.

Analytical techniques useful as detection methods are generally known in the art. For example, calorimetric, electrophoretic and radio-labelling techniques may be used (see Sambrook J., Fritsch E. F. & Maniatis T. (1989) Molecular cloning. A laboratory manual. 2 nd edn. Cold Spring Harbor Laboratory Press. Cold Spring Harbor, N.Y., the disclosure of which is hereby incorporated by reference).

Colorimetric techniques are generally preferred and may employ spectroscopic or visual verification of a colour change indicating a positive or negative test result. One skilled in the art will appreciate that other techniques may be employed as detection methods in the present invention.

The kit of the present invention may further comprise means for detecting the label, or such means may be separate from the kit.

By determining the presence of antibodies to ISAV, the presence or past presence of ISAV in a subject fish may be determined. Thus, the method of the present invention may be used to detect not only acute infection (one in which both the virus and antibody are present), but may also be used in cases wherein the antibody is present but there is no detectable virus, such as in ISAV vaccinated fish or fish that have recovered from ISAV infection. Prior art methods detect the virus directly and are therefore not useful in cases where only the antibody is present.

The method and kit of the present invention may be used for field application such as a routine laboratory test for detection of ISAV infection, and where vaccination is not performed the test can detect asymptomatic ISAV carriers to obtain a realistic estimate of the prevalence of ISAV infection, particularly since ISAV is notoriously difficult to isolate in certain circumstances. The test may also be used to assess ISAV vaccine efficacy before placing smolts in sea cages or for testing fish in sea cages to detect level of immunity from previous infection or vaccines.

The invention will now be described in more particularity having reference to particular examples and the appended drawings, which do not limit the invention.

### EXAMPLES

Materials and Methods

Preparation of Purified Virus Antigen:

Infectious salmon anaemia virus (ISAV) strains RPC/NB 990-02-4 and RPC/NB 970-877-2 were propagated in SHK-1 cell line as previously described (Kibenge et al., 2000a). Virus was concentrated using double ammonium sulphate precipitation followed by overnight dialysis against TNE (10 mM Tris-HCl, 0.1 M NaCl, 1 mM EDTA, pH 7.5) buffer. The dialysed virus was purified using Ficoll™ 400 step gradient (25% and 10%) and the extracted bands were pelleted through 20% sucrose. The virus pellet was dissolved in TNE buffer, quantitated and stored at −80° C. until used as the virus antigen to coat 96-well plates. The purity of the virus antigen was checked by electrophoresis on 12.5% SDS-polyacrylamide gels. A lysate of uninfected SHK-1 cells was used as a control for non-specific binding of the fish antibodies.

Fish Serum Samples:

Three groups of Atlantic salmon sera were used in this study. The first group consisted of a known positive Atlantic salmon serum field sample (identified on the basis of a positive virus neutralization test) and a known negative Atlantic salmon serum (obtained from experimental fish that had never been in sea water). These sera were used to optimize the assay reagents and the protocol. The second group were fish serum samples collected from surviving fish at 0, 2, 4 and 6 weeks following experimental infection with ISAV (Kibenge et al., 2000b). These were used to assess the analytical sensitivity of the ELISA system by end-point dilution analysis. The third group of Atlantic salmon sera consisted of 37 fish serum samples collected from various fish farms in the Bay of Fundy, Canada, on two separate occasions (17 field serum samples designated here as belonging to Group 3A and 20 as belonging to Group 3B). Two groups of Coho salmon sera were used for the study. The first group consisted of a known positive Coho salmon serum field sample (identified on the basis of a positive virus neutralization test) and a known negative Coho salmon serum (obtained from experimental fish that had never been in sea water). The second group consisted of 19 pools of Coho salmon serum samples from a clinically affected fish farm in Chile (provided by Aquagestation, Fundacion Chile, Puerto Montt, Chile). All samples were heat-inactivated at 560° C. prior to use.

Virus Neutralization (VN) Test:

The VN test was carried out on cell monolayers of the TO cell line (Wergeland & Jakobsen 2001) grown in 48-well culture plates. Cell monolayers were grown at room temperature (22° C.) in HMEM [Eagle's minimum essential medium containing Hank's salts] (BioWhittaker Inc.) supplemented with 292 μg ml⁻¹ L-glutamine (Sigma), 1% Non Essential Amino Acids (NEAA) (Sigma), 100 μg ml⁻¹ gentamicin (Sigma) and 10% foetal bovine serum (FBS). For maintenance medium, FBS was reduced to 5%. To set up a VN test, serial 2-fold dilutions of fish serum and an equal volume of virus suspension containing 100 TCID₅₀ of ISAV strain RPC/NB 990-02-4 were added to TO cell monolayers drained of medium and incubated at room temperature for 1 hr before addition of 500 μl of fresh maintenance medium to each well. After a further 10 days' incubation at 16° C., cultures were examined for cytopathic effects (CPE) to determine the VN test results.

Indirect ELISA System:

Different types of ELISA plates, coating antigen concentrations, blocking buffers, incubation periods and temperatures, and fish serum preparation were optimized during preliminary experiments. The optimal conditions were then used as described. The ELISA plates (Falcon Pro Bind Assay™ plates, VWR) were coated with 1.0 μg of virus antigen per well in 100 μl of 0.2 M sodium bicarbonate buffer, pH 9.6, and incubated overnight at 40° C. (Arkoosh & Kaattari 1990). In order to ascertain the analytical specificity of the ELISA system, an uninfected SHK-1 cell lysate was used as the negative (or irrelevant) antigen at a concentration of 10 μg/ml.

Following the overnight incubation, the plates were washed three times with Dulbecco's phosphate buffered saline (PBS) with 0.05% (v/v) Tween™ 20 (T-PBS) and unbound sites were blocked by adding to each well 100 μl of 3% normal goat serum in T-PBS and incubating at room temperature for 1 hr. The blocking medium was removed, and after washing as described above, fish serum samples in two-fold dilutions (from 1:10 to 1:5120) in T-PBS with 1% normal goat serum were added to the wells (100 μl per well) in triplicate and incubated at 16° C. for 1.5 hr (Arkoosh & Kaattari 1990). On each plate a row of “blank wells” was left to which T-PBS instead of fish serum was added. These “blank wells” were used to determine the background optical density (OD) of the ELISA system. The ELISA plates were then quickly washed three times followed by three 10-minute washes.

After the last wash, mouse monoclonal antibody IPA3D1 (Immuno-Precise Antibodies Ltd., Victoria, British Columbia, Canada) to Atlantic salmon immunoglobulin, diluted 1:500 in T-PBS with 1% normal goat serum, was added to all wells (100 μl per well) and incubated at 37° C. for 1 hr. For ELISA on Coho salmon serum samples, mouse monoclonal antibody IPA2C7 (Immuno-Precise Antibodies Ltd., Victoria, British Columbia, Canada) to Rainbow trout immunoglobulin, was used at a dilution of 1:1500 in T-PBS with 1% normal goat serum (in preliminary tests it was established that Mab IPA2C7 does not recognize Atlantic salmon immunoglobulins). The ELISA plates were washed as described above. Then 100 μl of goat anti-mouse IgG-1 alkaline phosphatase-conjugate (Cedarlane Laboratories Ltd., Hornby, Ontario, Canada) diluted 1:1500 in T-PBS with 1% goat serum was added to each well and the plates were incubated for 1 hr at 37° C. The plates were again washed to remove unbound conjugate, and 100 μl of the enzyme substrate p-nitrophenyl phosphate (Sigma) was added to each well.

The plates were incubated for 45 min at 37° C. in the dark to develop optimal colour and then the OD was read in an automatic microtitre ELISA reader (Spectra Max™ 340) at 405 nm wavelength. In some cases, incubation was continued overnight at 37° C. before taking a final reading. To determine the optimal incubation interval during which the OD reading would be proportional to the antibody concentration, readings were taken at 10 minute intervals for 4 hrs and a final reading after 24 hrs.

Indirect Competition ELISA:

A competitive assay for indirect ELISA was set up using a rabbit anti-ISAV serum (Kibenge et al., 2000a) diluted 1:200 as the first antibody added to the virus antigen. After blocking the wells with 3% goat serum and washing the plates, 100μl of rabbit anti-ISAV serum (1:200) was added to all the wells and incubated at 16° C. for 1.5 hr, then the plates were washed as described above, and known positive and negative Atlantic salmon sera were added to the wells and the rest of assay was carried out as described above. The OD reading in the plates that were incubated with rabbit anti-ISAV serum were compared with those assayed without the addition of rabbit anti-ISAV serum.

Reverse-Transcriptase Polymerase Chain Reaction (RT-PCR):

Fish tissue samples were examined for presence of ISAV nucleic acids by RT-PCR as previously described (Kibenge et al., 2000b). Briefly, viral RNA was extracted from 250 μl volumes of tissue homogenates using TRIZOL LS Reagent (Invitrogen Life Technologies) following the manufacturer's protocol.

The PCR primers targeting ISAV RNA segment 8 and consisting of 5′-GAA GAG TCA GGA TGC CAA GAC G-3′ (FA-3, sense) and 5′-GAA GTC GAT GAT CTG CAG CGA-3′ (RA-3, antisense) which yield a PCR product of 220 bp were used for detection of ISAV nucleic acids in fish tissue samples. One-step RT-PCR was carried out using the Titan™ One Tube RT-PCR System kit (Roche Molecular Biochemicals). The RT-PCR was performed in a PTC-200 DNA Engine Peltier thermal cycler (M J Research, Inc., Watertown, Mass., USA).

Cycling conditions consisted of one cycle of cDNA synthesis and pre-denaturation at 55° C. for 30 min and 94° C. for 2 min, followed by 40 cycles each consisting of denaturation at 94° C. for 30 sec, annealing at 61° C. for 45 sec, and extension at 72° C. for 90 sec, with a final extension at 72° C. for 10 min. PCR products were resolved by electrophoresis on a 2% agarose gel and visualized under 304 nm UV light after staining with ethidium bromide (Sambrook, Fritsch & Maniatis 1989).

Statistical Analysis:

The average OD of the blank wells on each ELISA plate was subtracted from the OD of each well on the plate. For each dilution of a serum sample, the results were reported as the corrected mean±standard error of the mean, and the difference between the virus antigen and cell antigen was compared using unpaired t-test. The end point for the assay was calculated from the mean OD plus 2 standard deviations of the positive control serum when reacted against the cellular antigens (i.e., 0.24 and 0.26 OD units for Atlantic salmon and Coho salmon, respectively, above activity against cellular antigens). A variation coefficiency was performed by evaluating results from replicates of known positive and negative Atlantic salmon sera in each plate within a run and between runs of the assay in order to determine the intraplate and interplate variations. The interassay and intrassay coefficiencies were less than 10% in both cases.

Results

Determination of Optimal Antigen Concentration:

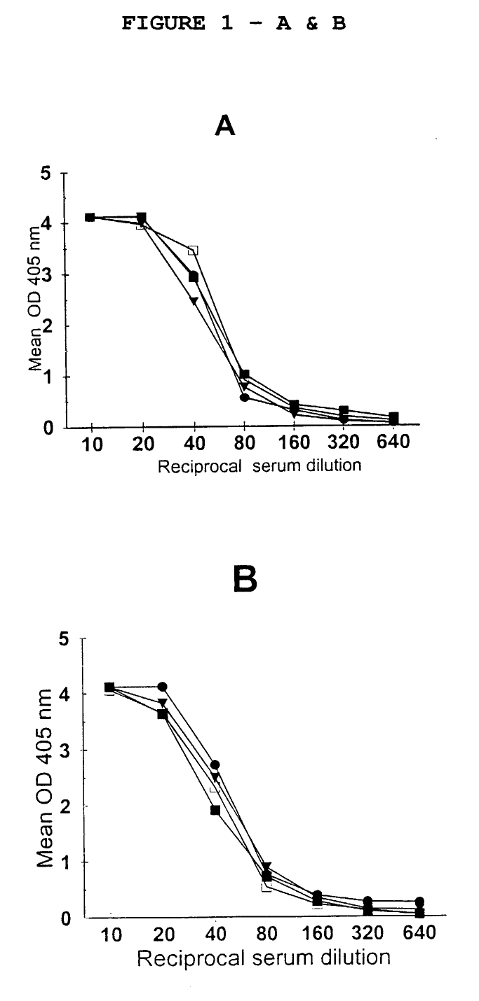

The optimal concentration of antigen adsorbed to the plate was determined by testing 2.5 μg, 5 μg, 7.5 μg, and 10 μg/ml of virus antigen using ISAV isolates RPC/NB 990-02-4 and RPC/NB 970-877-2. Isolate RPC/NB 970-877-2 is a CHSE-negative phenotype whereas RPC/NB-990-02-4 is a CHSE-positive phenotype (Kibenge et al., 2000a). As shown in FIG. 1, no difference was observed in the detection level with all four virus antigen concentrations within each isolate and among the two ISAV isolates. Moreover, use of a blank (i.e., no fish serum added) showed the test to have very low background activity for all antigen concentrations used. Therefore, the virus antigen prepared from isolate RPC/NB-990-02-4 was used at a concentration of 10 μg/ml (1.0 μg/well) in subsequent assays as the positive antigen.

Determination of Optimum Incubation Time for Colour Development:

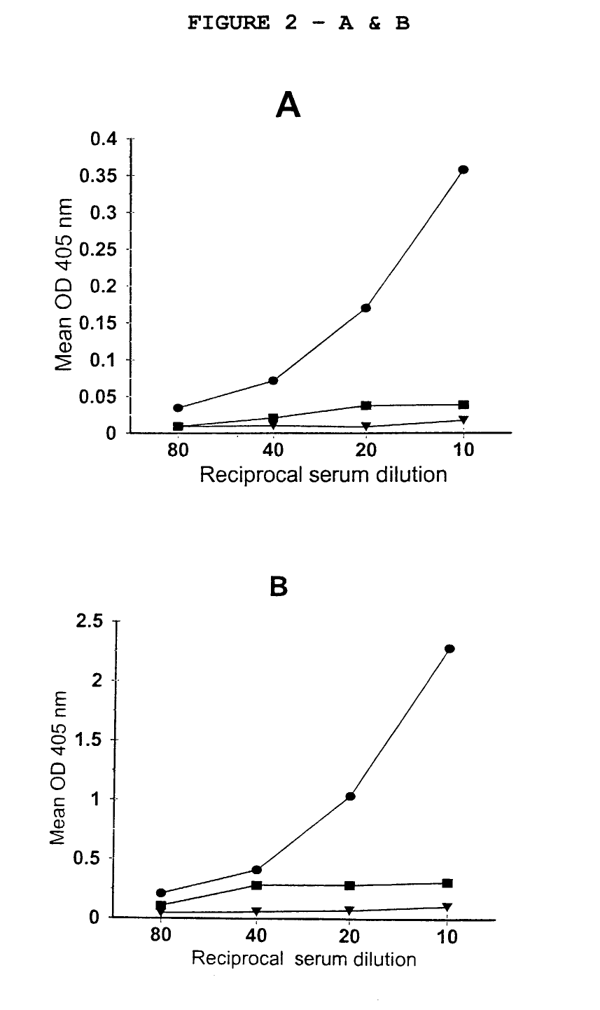

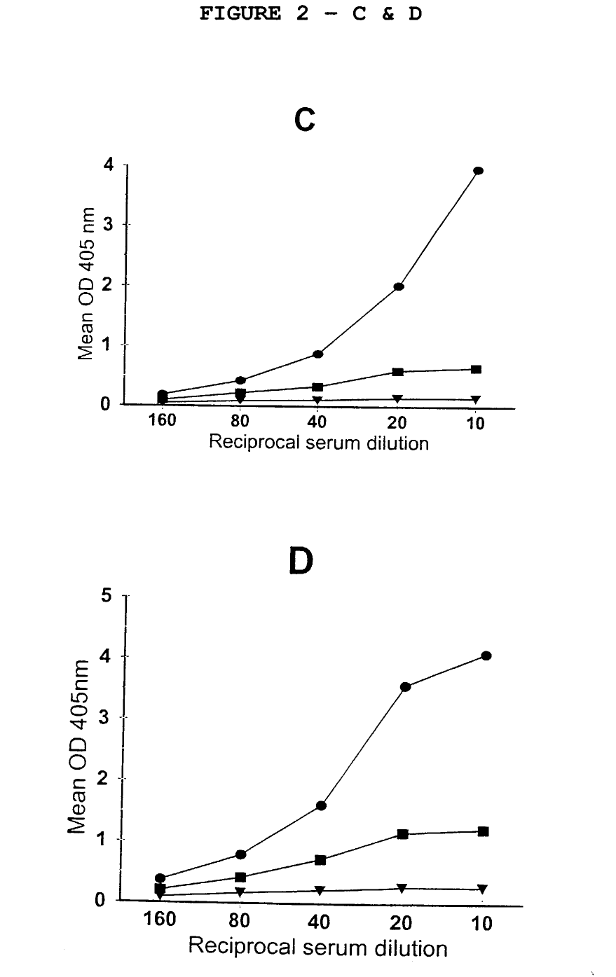

In order to determine the optimum incubation interval during which the OD reading would be proportional to the antibody concentration, readings were taken at 10 minute intervals for 4 hr and a final reading after 24 hr. FIG. 2 shows that the rate of colour development was proportional to the amount of antibody in the fish serum sample for at least 2 hr of incubation in samples with high antibody levels. In contrast, samples with low antibody levels showed little or no change in the rate of colour development after incubations of up to 4 hr. Consequently, all serum samples were read after 45 min and overnight incubations at 37° C. After 45 min of incubation, the antibody titer of the positive Atlantic salmon serum was 1:640 with a corrected mean O.D of 0.4±0.02 against the virus antigen compared to 0.0 O.D with the cellular antigens. The antibody titer detected with overnight incubation was 1:1280 with a corrected mean O.D of 0.35±0.04 against the virus antigen compared to 0.0 O.D with the cellular antigens (Table 1).

Indirect Competition ELISA:

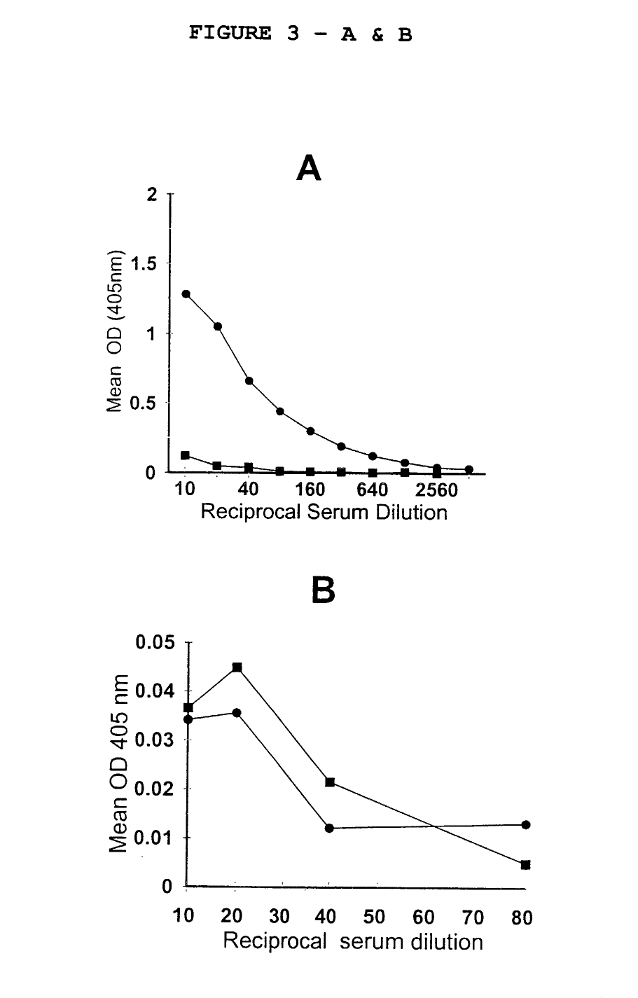

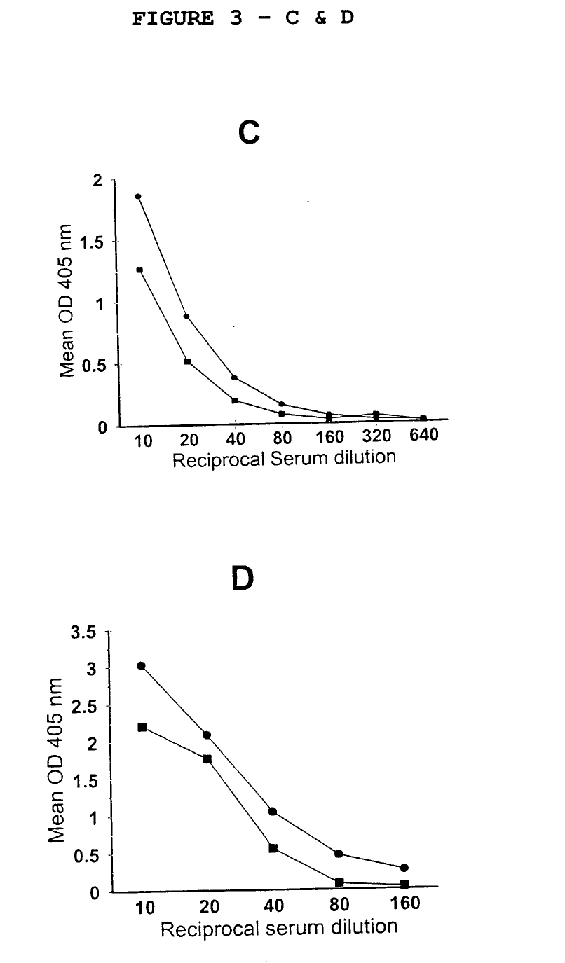

To confirm the specificity of the indirect ELISA system for fish antibodies to ISAV, a competitive assay was set up whereby the antigen was first reacted to rabbit anti-ISAV serum before adding the fish serum. The results on both positive and negative fish sera when reacted against viral and cellular antigens are shown in FIG. 3. The rabbit anti-ISAV serum blocked only the positive fish serum when reacted against virus antigen as shown by the almost negligible OD readings.

ELISA ISAV Antibody Levels in Serum of Atlantic Salmon Experimentally Inoculated with ISAV:

Atlantic salmon serum from uninfected control fish from virus-infected fish at 0, 2, 4 and 6 weeks post inoculation (wpi) were assayed for the presence of ISAV antibodies together with the positive and negative control Atlantic salmon sera. The use of fish serum without heat-inactivation resulted in high non-specific reactions, particularly with the cellular (negative) antigen (data not shown). These non-specific reactions were completely eliminated if samples were heat-inactivated at 56° C. for 30 minutes. Therefore, all serum samples were heat-inactivated prior to use.

Using the calculated end point of 0.24 OD units above the activity against the cellular antigen, ISAV antibodies in the virus-infected experimental fish first appeared at 6 wpi (Table 1). An antibody titer of 1:20 was recorded after incubation for 45 minutes with the substrate, which rose to 1:160 after overnight incubation. The mean OD readings at these serum dilutions were significantly higher with the virus antigen than with the cellular (negative) antigen (p<0.05), indicating that this reaction is specific to the virus antigen and that this indirect ELISA can be used to assess the humoral response of recently infected fish.

The assay was set up such that a known positive control Atlantic salmon serum was ran on each plate in order to ensure that the data obtained from one plate was comparable to the data obtained from other plates (Tijssen 1985). A variation coefficiency was performed on mean O.Ds of this positive fish serum to determine the intraplate and interplate variations. Testing of the positive control fish serum to evaluate intraplate and interplate variations indicated adequate repeatability (6% coefficients of variation).

TABLE 1
ELISA ISAV Antibody Levels in Fish Experimentally Infected with ISAV

| | Fish serum samples | | | | | | | |
|---|---|---|---|---|---|---|---|---|
| | Control negative serum | | Control positive serum | | 4 weeks p.i. | | 6 weeks p.i. | |
| Dilution | Virus¹ | Cells² | Virus¹ | Cells² | Virus¹ | Cells² | Virus¹ | Cells² |
| 0.04861 | 0.009 ± 0.001 | 0.02 ± 0.002 | 4.059 ± 0 | 0.2 ± 0.02 | 0.294 ± 0.017 | 0.21 ± 0.081 | 2.84 ± 0.13* | 0.31 ± 0.079 |
| 0.05556 | 0.006 ± 0.002 | 0.001 ± 0.001 | 4.059 ± 0 | 0.152 ± 0.003 | 0.200 ± 0.020 | 0.145 ± 0.033 | 1.8 ± 0.14* | 0.27 ± 0.0124 |
| 0.06944 | | | 3.63 ± 0.31 | 0.142 ± 0.001 | 0.111 ± 0.018 | 0.072 ± 0.002 | 1.124 ± 0.115* | 0.113 ± 0.0241 |
| 1:80 | | | 2.21 ± 0.29 | 0.057 ± 0.003 | 0.063 ± 0.012 | 0.043 ± 0.008 | 0.556 ± 0.035* | 0.095 ± 0.054 |
| 1:160 | | | 1.451 ± 0.11 | 0.0512 ± 0.001 | 0.042 ± 0.015 | 0.038 ± 0.024 | 0.31 ± 0.016* | 0.061 ± 0.038 |
| 1:320 | | | 0.703 ± 0.1 | 0.016 ± 0.002 | 0.024 ± 0.006 | 0.021 ± 0.006 | 0.151 ± 0.014 | 0.036 ± 0.004 |
| 1:640 | | | 0.476 ± 0.04 | | 0.039 ± 0.018 | 0.024 ± 0.003 | 0.095 ± 0.025 | |
| 1:1280 | | | 0.35 ± 0.04 | | | | | |
| 1:2560 | | | 0.176 ± 0.02 | | | | | |
| 1:5120 | | | 0.167 ± 0.001 | | | | | |

¹ISAV RPC/NB 990-002(4) at concentration of 1 μg/well
²uninfected SHK-1 cell lysate (1 μg/well)
The reported results are corrected mean OD₄₀₅ ± STD

ELISA ISAV Antibody Levels in Field Atlantic Salmon Serum Samples from New Brunswick:

Two groups of field Atlantic salmon serum samples were tested for the presence of ISAV antibodies. Seventeen samples were collected as Group 3A and 20 as Group 3B, from farmed Atlantic salmon on various fish farms in the Bay of Fundy, Canada. In Group 3A, the fish sampled were also tested for ISAV by RT-PCR using primers to RNA segment 8 as previously described (Kibenge et al., 2000a). For all field samples, the calculated O.Ds with either virus or cellular antigens were very low after 45 min of incubation (data not shown), indicating the lower levels of antibody to ISAV in such samples.

In fish that tested positive for ISAV by RT-PCR, the calculated O.Ds for cellular antigens remained low (i.e., <0.24 O.D) even following overnight incubations, allowing the detection of an ISAV-specific antibody response (Table 2). When the ELISA results were compared with the RT-PCR results on the same fish, ELISA was able to positively identify 71% of the RT-PCR ISAV positive fish (71% sensitivity), and all RT-PCR negative fish were also negative in the indirect ELISA (100% specificity). This ELISA system was also able to differentiate between ISAV antibody-positive and negative fish sera in Group 3B.

Interestingly, in the Group 3A field samples, the sera from fish that were negative for ISAV by RT-PCR had high corrected mean O.Ds for both the virus and cellular antigens particularly at low serum dilutions (Table 2). In these samples, the difference in the O.D between the virus and cellular antigens was invariably less than 0.24 O.D units, except for sample B at 1:80 dilution (Table 2). A similar observation was made for the ELISA-negative field sera in Group 3B (Table 3). Thus, the ELISA-negative Atlantic salmon tended to have an elevated non-specific antibody response which might be suggestive of either chronic infection or resistance to ISAV infection.

TABLE 2
ELISA ISAV Antibody Levels in Group 3A Field Serum Samples

| | Serum dilution | | | | | |
|---|---|---|---|---|---|---|
| Fish ID/Ag¹ | 1:10 | 1:20 | 1:40 | 1:80 | 1:160 | 1:320 |
| A**/Virus | 0.35 ± 0.027 | 0.191 ± 0.011 | 0.154 ± 0.002 | 0.104 ± 0.001 | 0.015 ± 0.0 | |
| A**/SHK-1 | 0.39 ± 0.028 | 0.146 ± 0.005 | 0.194 ± 0.014 | 0.058 ± 0.001 | | |
| B**/Virus | 1.08 ± 0.054 | 0.79 ± 0.054 | 0.08 ± 0.023 | 0.49 ± 0.053 | 0.28 ± 0.01 | 0.181 ± 0.012 |
| B**/SHK-1 | 1.40 ± 0.72 | 0.69 ± 0.011 | 0.48 ± 0.01 | 0.24 ± 0.005 | 0.204 ± 0.041 | 0.106 ± 0.006 |
| D**/Virus | 0.67 ± 0.014 | 0.481 ± 0.007 | 0.234 ± 0.001 | 0.185 ± 0.013 | 0.13 ± 0.013 | 0.057 ± 0.01 |
| D**/SHK-1 | 1.18 ± 0.045 | 0.73 ± 0.001 | 0.54 ± 0.03 | 0.4 ± 0.013 | 0.23 ± 0.001 | 0.121 ± 0.013 |
| E*/Virus | 1.36 ± 0.061 | 0.911 ± 0.018 | 0.623 ± 0.007 | 0.417 ± 0.05 | 0.237 ± 0.02 | 0.162 ± 0.001 |
| E*/SHK-1 | 0.155 ± 0.004 | 0.0834 ± 0.01 | 0.086 ± 0.002 | 0.075 ± 0.01 | 0.024 ± 0.007 | 0.014 ± 0.004 |
| F*/Virus | 2.22 ± 0.138 | 1.51 ± 0.1 | 0.874 ± 0.028 | 0.411 ± 0.026 | 0.3 ± 0.021 | 0.139 ± 0.003 |
| F*/SHK-1 | 0.003 ± 0.001 | 0.038 ± 0.003 | 0.012 ± 0.001 | 0.0197 ± 0.00 | 0.025 ± 0.004 | |
| H*/Virus | 2.58 ± 0.17 | 1.72 ± 0.1 | 0.82 ± 0.011 | 0.49 ± 0.006 | 0.25 ± 0.01 | 0.149 ± 0.01 |
| H*/SHK-1 | 0.097 ± 0.007 | 0.046 ± 0.005 | 0.041 ± 0.002 | 0.018 ± 0.001 | 0.007 ± 0.001 | |
| I*/Virus | 4.07 ± 0.0 | 4.07 ± 0.0 | 4.07 ± 0.0 | 3.94 ± 0.18 | 3.08 ± 0.12 | 2.14 ± 0.3 |
| I*/SHK-1 | 0.241 ± 0.08 | 0.058 ± 0.026 | 0.02 ± 0.006 | | | |
| J*/Virus | 2.04 ± 0.126 | 1.25 ± 0.042 | 0.643 ± 0.007 | 0.324 ± 0.003 | 0.133 ± 0.007 | 0.085 ± 0.002 |
| J*/SHK-1 | 0.137 ± 0.013 | 0.092 ± 0.005 | 0.059 ± 0.001 | 0.038 ± 0.001 | 0.026 ± 0.002 | 0.022 ± 0.003 |
| L**/Virus | 0.12 ± 0.014 | 0.093 ± 0.004 | 0.067 ± 0.002 | 0.03 ± 0.0 | | |
| L**/SHK-1 | ND | ND | ND | ND | | |
| M**/Virus | 0.28 ± 0.045 | 0.145 ± 0.023 | | | | |
| M**/SHK-1 | 0.11 ± 0.001 | 0.027 ± 0.008 | | | | |
| N^{$}**/Virus | 0.46 ± 0.019 | 0.31 ± 0.015 | 0.22 ± 0.015 | 0.171 ± 0.001 | 0.123 ± 0.006 | 0.075 ± 0.005 |
| N^{$}**/SHK-1 | 0.432 ± 0.002 | 0.373 ± 0.001 | 0.31 ± 0.01 | 0.133 ± 0.004 | 0.074 ± 0.005 | 0.06 ± 0.003 |
| O*/Virus | 0.147 ± 0.001 | 0.093 ± 0.002 | 0.063 ± 0.005 | 0.044 ± 0.003 | | |
| O*/SHK-1 | 0.063 ± 0.003 | 0.043 ± 0.001 | 0.056 ± 0.002 | 0.024 ± 0.004 | | |
| P**/Virus | 0.156 ± 0.021 | 0.1 ± 0.016 | 0.13 ± 0.014 | 0.12 ± 0.001 | 0.151 ± 0.02 | 0.11 ± 0.03 |
| P**/SHK-1 | 0.32 ± 0.03 | 0.22 ± 0.03 | 0.25 ± 0.004 | 0.1 ± 0.023 | 0.164 ± 0.0 | 0.058 ± 0.008 |
| Q**/Virus | 0.66 ± 0.034 | 0.629 ± 0.04 | 0.4 ± 0.06 | 0.29 ± 0.002 | 0.172 ± 0.01 | 0.07 ± 0.002 |
| Q**/SHK-1 | 0.64 ± 0.022 | 0.41 ± 0.003 | 0.373 ± 0.03 | 0.168 ± 0.02 | 0.10 ± 0.01 | 0.033 ± 0.004 |
| R**/Virus | 0.37 ± 0.046 | 0.164 ± 0.002 | 0.121 ± 0.01 | 0.055 ± 0.001 | 0.034 ± 0.003 | 0.031 ± 0.001 |
| R**/SHK-1 | 0.64 ± 0.03 | 0.37 ± 0.01 | 0.22 ± 0.008 | 0.12 ± 0.012 | 0.053 ± 0.003 | 0.076 ± 0.007 |
| S**/Virus | 0.156 ± 0.015 | 0.142 ± 0.002 | 0.078 ± 0.004 | 0.066 ± 0.006 | 0.033 ± 0.006 | |
| S**/SHK-1 | 0.241 ± 0.029 | 0.122 ± 0.07 | 0.085 ± 0.005 | 0.02 ± 0.004 | 0.031 ± 0.027 | |
| T*/Virus | 0.165 ± 0.013 | 0.095 ± 0.007 | 0.083 ± 0.007 | 0.076 ± 0.01 | 0.026 ± 0.005 | 0.079 ± 0.004 |
| T*/SHK-1 | 0.095 ± 0.007 | 0.083 ± 0.007 | 0.076 ± 0.01 | 0.026 ± 0.005 | 0.08 ± 0.004 | 0.12 ± 0.02 |

¹Ag denotes Antigen (Virus or SHK-1)
*= positive by RT-PCR;
**= negative by RT-PCR;
^{$}= not vaccinated
ND = OD below background

TABLE 3
ELISA ISAV Antibody Levels in Group 3B Field Serum Samples

| | Serum dilution | | | |
|---|---|---|---|---|
| | Virus antigen | | SHK-1 cells | |
| Fish serum # | 1:10 | 1:20 | 1:10 | 1:20 |
| 1 | 0.573 ± 0.036 | 0.38 ± 0.057 | 1.04 ± 0.057 | 0.454 ± 0.032 |
| 2* | 0.352 ± 0.020 | 0.235 ± 0.001 | 0.07 ± 0.057 | 0.089 ± 0.002 |
| 3 | 0.14 ± 0.005 | 0.11 ± 0.009 | 0.414 ± 0.038 | 0.255 ± 0.01 |
| 4* | 0.625 ± 0.011 | 0.526 ± 0.033 | 0.133 ± 0.025 | 0.105 ± 0.021 |
| 5 | 1.07 ± 0.037 | 1.35 ± 0.092 | 0.911 ± 0.03 | 0.852 ± 0.03 |
| 6 | 0.307 ± 0.022 | 0.484 ± 0.01 | 0.52 ± 0.034 | 0.258 ± 0.002 |
| 7 | 0.052 ± 0.004 | 0.036 ± 0.001 | 0.266 ± 0.024 | 0.121 ± 0.011 |
| 8* | 2.96 ± 0.113 | 1.69 ± 0.13 | 0.323 ± 0.044 | 0.221 ± 0.011 |
| 9 | 0.177 ± 0.017 | 0.104 ± 0.001 | 2.03 ± 0.18 | 0.991 ± 0.069 |
| 10 | 0.062 ± 0.005 | 0.064 ± 0.004 | 0.461 ± 0.013 | 0.274 ± 0.0004 |
| 11 | 0.303 ± 0.007 | 0.186 ± 0.014 | 2.97 ± 0.12 | 1.63 ± 0.018 |
| 12 | 0.046 ± 0.004 | 0.04 ± 0.003 | 0.095 ± 0.001 | 0.044 ± 0.003 |
| 13 | 0.320 ± 0.024 | 0.201 ± 0.02 | 0.146 ± 0.024 | 0.153 ± 0.002 |
| 14 | 0.036 ± 0.001 | 0.029 ± 0.002 | 0.044 ± 0.001 | 0.021 ± 0.0004 |
| 15 | 0.363 ± 0.002 | 0.322 ± 0.006 | 0.261 ± 0.006 | 0.268 ± 0.007 |
| 16 | 0.222 ± 0.018 | 0.1 ± 0.016 | 0.666 ± 0.048 | 0.229 ± 0.01 |
| 17 | 0.762 ± 0.03 | ND | 0.875 ± 0.06 | ND |
| 18 | 0.136 ± 0.013 | 0.138 ± 0.001 | 0.109 ± 0.001 | 0.053 ± 0.001 |
| 19 | 0.061 ± 0.0 | 0.068 ± 0.01 | 0.655 ± 0.09 | 0.143 ± 0.014 |
| 20 | 4.052 ± 0.049 | 3.82 ± 0.26 | 4.083 ± 0.0 | 4.083 ± 0.0 |

ND = not done
*= ELISA positive

ISAV Antibody in Coho Salmon Serum Samples from Chile:

Identification of ELISA-positive and negative Coho salmon sera was based on the cut off of 0.263 OD units above activity against cellular antigen established using a known positive control. Statistical analysis using the unpaired t-test between reactivity of the serum sample to the virus antigen and to the cellular antigen. Table 4 summarizes the ELISA results on the Coho salmon samples. Overall, the Coho salmon serum pool samples had high reactivity to ISAV antigen. Eleven (57.9%) of 19 samples were identified as ELISA ISAV antibody positive at both 1:10 and 1:20 serum dilutions. Coho salmon serum pool #15 was positive only at 1:10 dilution whereas #14 was positive only at 1:20 dilution (Table 4). The reactivity of Coho salmon serum pool #14 at 1:10 dilution was probably a prozone effect error.

The RT-PCR testing for ISAV was also performed on this group of fish using heart, liver and kidney tissue pooled samples. All six liver pools tested were positive for ISAV as were four of six heart pools and kidney pools. However, because the sera and tissues were pooled samples, no correlation could be drawn between the antibody ELISA and RT-PCR results.

TABLE 4
ELISA ISAV Antibody Levels in Coho Salmon Serum Samples from
Chile

| | Serum dilution | | | |
|---|---|---|---|---|
| | ISAV antigen | | SHK-1 cells | |
| Fish serum # | 1:10 | 1:20 | 1:10 | 1:20 |
| 1* | 0.739 ± 0.047 | 0.762 ± 0.006 | 0.234 ± 0.018 | 0.184 ± 0.007 |
| 2 | 0.574 ± 0.052 | 0.472 ± 0.003 | 0.315 ± 0.011 | 0.308 ± 0.022 |
| 3 | 1.315 ± 0.131 | 0.832 ± 0.024 | 1.270 ± 0.092 | 0.759 ± 0.055 |
| 4 | 0.305 ± 0.029 | 0.299 ± 0.028 | 0.167 ± 0.012 | 0.132 ± 0.020 |
| 5* | 2.009 ± 0.187 | 1.388 ± 0.064 | 1.541 ± 0.107 | 0.637 ± 0.063 |
| 6 | 0.395 ± 0.015 | 0.269 ± 0.022 | 0.202 ± 0.001 | 0.100 ± 0.015 |
| 7* | 0.840 ± 0.053 | 0.802 ± 0.051 | 0.458 ± 0.034 | 0.325 ± 0.006 |
| 8* | 1.046 ± 0.131 | 0.838 ± 0.071 | 0.558 ± 0.036 | 0.415 ± 0.062 |
| 9 | 0.220 ± 0.007 | 0.262 ± 0.012 | 0.142 ± 0.004 | 0.181 ± 0.017 |
| 10* | 1.818 ± 0.126 | 1.804 ± 0.047 | 0.914 ± 0.075 | 0.566 ± 0.030 |
| 11* | 0.598 ± 0.028 | 0.412 ± 0.023 | 0.290 ± 0.036 | 0.082 ± 0.020 |
| 12* | 1.651 ± 0.116 | 1.242 ± 0.068 | 0.882 ± 0.073 | 0.695 ± 0.030 |
| 13* | 1.002 ± 0.145 | 0.869 ± 0.071 | 0.234 ± 0.014 | 0.430 ± 0.010 |
| 14 | 0.388 ± 0.044 | *0.481 ± 0.035 | 0.158 ± 0.026 | 0.196 ± 0.021 |
| 15 | *0.879 ± 0.075 | 0.652 ± 0.033 | 0.273 ± 0.038 | 0.560 ± 0.034 |
| 16* | 3.456 ± 0.076 | 1.942 ± 0.152 | 2.259 ± 0.154 | 1.272 ± 0.076 |
| 17 | 0.382 ± 0.024 | 0.360 ± 0.003 | 0.374 ± 0.019 | 0.197 ± 0.012 |
| 18* | 0.846 ± 0.021 | 0.812 ± 0.021 | 0.519 ± 0.018 | 0.450 ± 0.052 |
| 19* | 0.682 ± 0.022 | 0.666 ± 0.001 | 0.183 ± 0.020 | 0.129 ± 0.013 |
| CS¹ positive | 1.002 ± 0.145 | 0.869 ± 0.071 | 0.234 ± 0.014 | 0.430 ± 0.010 |
| CS negative | 0.498 ± 0.019 | 0.441 ± 0.033 | 0.259 ± 0.024 | 0.271 ± 0.003 |

*ELISA positive
¹CS denotes Control Coho salmon serum

Discussion

The ELISA system described herein is capable of detecting antibody to ISAV in fish as demonstrated by application to serum samples from farmed Atlantic salmon in the Bay of Fundy, New Brunswick, Canada, and to serum samples from farmed Coho salmon in Chile. Although previous studies reported increased resistance of Atlantic salmon to ISAV upon re-infection or after passive immunization with serum from fish that had recovered from ISA (Falk & Dannevig 1995), or following vaccination with inactivated virus (Jones, Mackinnon & Salonius 1999), antibody levels in such fish were not determined. Others have tried and failed to determine ISAV antibody titers against ISAV in sera from vaccinated Atlantic salmon (Brown, Sperker, Clouthier & Thornton, 2000).

The ELISA system of this example uses purified virus antigen as coating antigen and shows two different types of antibody response to ISAV by field fish. Naturally infected Atlantic salmon carrying ISAV detectable by RT-PCR had a specific antibody response to ISAV suggestive of a recent infection whereas those that were virus negative by RT-PCR had an elevated non-specific antibody response suggestive of chronic infection or resistance to ISAV. Serum from experimental fish up to 6 wpi did not show the elevated non-specific antibody response.

Comparison of the mean optical density (OD) of the known positive control fish serum between the virus antigen and the SHK-1 cell lysate antigen using unpaired t-test showed the test to be highly specific for antibodies to ISAV in both Atlantic salmon and Coho salmon. This is the first report demonstrating specific circulating antibody in serum of fish exposed to ISAV. The use of fish serum without heat-inactivation may result in high non-specific reactions, particularly with the cellular (negative) antigen. It was our observation that these non-specific reactions were completely eliminated if samples were heat-inactivated at 56° C. for 30 min. Dixon, Hattenberger-Baudouy & Way (1994) inactivated carp serum for 30 min at 45° C. before use in virus neutralization and competitive ELISA. However, it is known that the activity of IgM is destroyed or reduced by heat, and since the predominant antibody in fish is IgM (Denzin & Staak, 2000), it could be argued that its activity may have been somehow affected. Our data suggest that heat-inactivation of the fish sera at 56° C. for 30 min contributed to the specificity of the assay.

Analytical specificity for the test was assessed by use of cellular antigens (uninfected SHK-1 cell lysate) since these were considered the most likely source for cross-reactions because their content in the virus antigen preparation used would depend on the degree of purity of the virus. The purity of the preferred virus antigen was demonstrated by the fact that it successfully attached to the ELISA plate over a wide range of concentrations.

While the known positive serum and known negative serum controls did not react with the uninfected SHK-1 cell lysate antigens, it was our experience that field sera from fish that tested negative for ISAV by RT-PCR reacted relatively strongly with both the virus and the cellular antigens, particularly at low serum dilutions (Table 2). Therefore, to differentiate between the specific antibodies to ISAV and the non-specific antibody response, the fish sera were titrated and the end-point value was calculated for each individual serum by comparing the difference in mean O.D between the virus antigen and the cellular antigens. Antibody titers were calculated using a cut-off of 0.24 OD units above activity against cellular antigens. On this basis, the ELISA system was able to differentiate between an ISAV-specific antibody response (suggestive of a recent infection) and the non-specific antibody response to ISAV (suggestive of chronic infection or resistance to ISAV) in Atlantic salmon.

One of the reasons fish serology has not been widely used in infectious disease diagnosis is because fish immunoglobulins are predominantly of IgM isotype which is generally of relatively low specificity (Denzin & Staak 2000). In the present example, an indirect competition ELISA was used to confirm specificity of the ELISA system for fish antibodies to ISAV antigens as demonstrated by the ability of rabbit anti-ISAV serum to specifically block the reactivity of the positive fish serum against the virus antigen.

We interpreted the presence of specific antibody to ISAV in farmed Coho salmon in Chile as evidence of infection with this virus or a cross-reacting but as yet unidentified virus in Coho salmon. ISAV has previously been isolated from farmed Coho salmon in Chile (Kibenge et al., 2001). The tissue samples of the fish that were the source of the serum were also tested by RT-PCR for the presence of ISAV. All six liver pools and four of six heart pools and kidney pools tested were positive for ISAV. However, because the sera and tissues were pooled samples, no correlation could be drawn between the antibody ELISA and RT-PCR results. We believe that the presence of anti-ISAV antibodies in marine-farmed Coho salmon in Chile where vaccination for ISAV is not practiced, is further unequivocal evidence of ISAV in farmed fish in Chile.

Examination of sera from surviving Atlantic salmon following experimental infection with ISAV showed that specific antibody was present beginning at 6 weeks post infection indicating that the assay could be used as a routine laboratory test for detection of ISAV infection, particularly when the virus is notoriously difficult to isolate from clinical specimens such as ISAV in farmed Coho salmon in Chile (Kibenge et al., 2001), and where vaccination is not performed such as in wild fish, the test can detect asymptomatic ISAV carriers.

Therefore, it is apparent that the ELISA system has potential for field application such as a routine laboratory test for detection of ISAV infection, and where vaccination is not performed the test can detect asymptomatic ISAV carriers to obtain a realistic estimate of the prevalence of ISAV infection, particularly since ISAV is notoriously difficult to isolate in certain circumstances. The elevated non-specific antibody response in ELISA-negative field fish suggests that the test can also be used to assess ISAV vaccine efficacy before placing smolts in sea cages or for testing fish in sea cages to detect level of immunity from previous infection or vaccines.

The invention being thus described, it is apparent to one skilled in the art that variations and modifications are possible and that such variations and modifications are intended to be included within the scope of the invention.

### REFERENCES

Anonymous. (2000a). ISA hits the Faroes, Fish Farming International 27 (5):47.

Anonymous. (2000b). Lapointe G. Mar. 16, 2001, Press Release by Commissioner, Maine Dept. of Marine Resources.

Anonymous: OIE (2000c). Manual of Standards for Diagnostic Tests and Vaccines. Fourth Edition.

Arkoosh M. R. & Kaattari S. L. (1990) Quantitation of fish antibody to a specific antigen by an enzyme-linked immunosorbent assay (ELISA). In: Techniques in Fish Immunology. (J. S. Stolen, T. C. Fletcher, D. P. Anderson, B. S. Roberson, W. B. van Muiswinkel, eds). Chapter 3. SOS Publications, Fair Haven, N.J.

Bouchard D, Keleher W, Opitz H M, Blake S, Edwards K C, Nicholson B L (1999). Isolation of infectious salmon anaemia virus (ISAV) from atlantic salmon in New Brunswick, Canada. Diseases of Aquatic Organisms 35, 131-137.

Brown L. L., Sperker S. A., Clouthier S. & Thornton J. C. (2000) Development of a vaccine against infectious salmon anaemia virus (ISAV). Bulletin of Aquaculture Association of Canada 100, 4-7.

Byrne P. J., MacPhee D. D., Ostland V. E., Johnson G. & Ferguson H. W. (1998) Haemorrhagic kidney syndrome of Atlantic salmon Salmo salar L. Journal of Fish Diseases 21, 81-91.

Dannevig B. H., Falk K. & Namork E. (1995) Isolation of the causal virus of infectious salmon anaemia (ISA) in a long-term cell line from Atlantic salmon head kidney. Journal of General Virology 76, 1353-1359.

Denzin N. & Staak C. (2000) Fish immunoglobulin—A sero-diagnosticians perspective. Bulletin of European Association of Fish Pathologists 20, 60-64.

Dixon P. F., Hattenberger-Baudouy A. -M. & Way K. (1994) Detection of carp antibodies to spring viraemia of carp virus by a competitive immunoassay. Diseases of Aquatic Organisms 19, 181-186.

Du Pasquier L. (1982) Antibody diversity in lower vertebrates—why is it so restricted? Nature 296, 311-313.

Evensen Ø., Thorud K. E. & Olsen Y. A. (1991) A morphological study of gross and light microscopic lesions of infectious anaemia in Atlantic salmon (Salmo salar). Research in Veterinary Science 51,215-222.

Falk K. & Dannevig B. H. (1995) Demonstration of a protective immune response in infectious salmon anaemia (ISA)-infected Atlantic salmon Salmo salar. Diseases of Aquatic Organisms 21, 1-5.

Falk K., Namork E., Rimstad E., Mjaaland S. & Dannevig B. H. (1997) Characterization of infectious salmon anaemia virus, an orthomyxo-like virus isolated from Atlantic salmon (Salmo salar L.). Journal of Virology 71, 9016-9023.

Falk K., Namork E. & Dannevig B. H. (1998) Characterization and applications of a monoclonal antibody against infectious salmon anaemia virus. Diseases of Aquatic Organisms 34, 77-85.

Jones S. R. M., Mackinnon A. M. & Salonius K. (1999) Vaccination of fresh-water-reared Atlantic salmon reduces mortality associated with infectious salmon anaemia virus. Bulletin of European Association of Fish Pathologists 19, 98-101.

Kibenge F. S. B., Lyaku J. R., Rainnie D. & Hammell L. K. ( 2000a) Growth of infectious salmon anaemia virus in CHSE-214 cells and evidence for phenotypic differences between virus strains. Journal of General Virology 81, 143-150.

Kibenge F. S. B., Whyte S. K., Hammell K. L., Rainnie D., Kibenge M. T. & Martin C. K. (2000b). A dual infection of infectious salmon anaemia (ISA) virus and a togavirus-like virus in ISA of Atlantic salmon Salmo salar in New Brunswick, Canada. Diseases of Aquatic Organisms 42, 11-15.

Kibenge F. S. B., Gárate O. N., Johnson G., Arriagada R., Kibenge M. J. T. & Wadowska D. (2001). Isolation and identification of infectious salmon anaemia virus (ISAV) from Coho salmon in Chile. Diseases of Aquatic Organisms 45, 9-18.

Lovely J. E., Dannevig B. H., Falk K., Hutchin L., MacKinnon A. M., Melville K. J., Rimstad E. & Griffiths S. G. (1999) First identification of infectious salmon anaemia virus in North America with haemorrhagic kidney syndrome. Diseases of Aquatic Organisms 35, 145-148.

Manning, M J. (1994) Fishes. In: Immunology: A comparative approach (R. J. Turner, ed.), pp.69-100. John Wiley & Sons.

Mjaaland S., Rimstand E., Falk K., Dannevig B. H. (1997) Genomic characterization of the virus causing infectious salmon anaemia in Atlantic salmon (Salmo salar L) :an orthomyxo-virus-like virus in a teleost. Journal of Virology 71, 7681-7686.

Mullins J. G., Groman D. & Wadwoska D. (1998) Infectious salmon anaemia in salt water Atlantic salmon (Salmo salar L.) In New Brunswick, Canada. Bulletin of European Association of Fish Pathologists 11, 110-114.

Nylund A., Alexandersen S., Løvik P. & Jakobsen P. (1994) The response of brown trout (Salmo trutta L.) to repeated challenge with infectious salmon anaemia (ISA). Bulletin of European Association of Fish Pathologists 14, 167-170.

Nylund A., Alexandersen S., Rolland J. B. & Jakobsen P. (1995) Infectious salmon anaemia virus (ISAV) in brown trout. Journal of Aquatic Animal Health 7,236-240.

Nylund A., Kvenseth A. M., Krossøy B. & Hodneland K. (1997) Replication of the infectious salmon anaemia virus (ISAV) in rainbow trout, Oncorhynchus mykiss (Walbaum). Journal of Fish Diseases 20,275-279.

Rimstad E., Falk K., Mikalsen A. B. & Teig A. (1999) Time course tissue distribution of infectious salmon anaemia virus in experimentally infected Atlantic salmon Salmo salar. Diseases of Aquatic Organisms 36, 107-112.

Ritchie R. J., Cook M., Mellville K., Simard N., Cusack R. & Griffiths, S. (2001) Identification of infectious salmon anaemia virus (ISAV) in Atlantic salmon from Nova Scotia (Canada): evidence for carrier fish and strain differences. Diseases of Aquatic Organisms 44, 171-178.

Rodger H. D. & Richards R. H. (1998) Haemorrhagic smolt syndrome: a severe anaemic condition in farmed salmon in Scotland. Veterinary Record 142, 538-541.

Sambrook J., Fritsch E. F. & Maniatis T. (1989) Molecular cloning. A laboratory manual. 2^{nd} edn. Cold Spring Harbor Laboratory Press. Cold Spring Harbor, N.Y.

Thorud K. & Djupvik H. O. (1988) Infectious anaemia in Atlantic Salmon (Salmo salar L.). Bulletin of European Association of Fish Patholologists 18, 109-111.

Tijssen P. (1985) Laboratory Techniques in Biochemistry and Molecular Biology. In: Practice and Theory of Enzyme Immunoassays. (ed. By R H. Burdon & P H. van Knippenberg), Chapter 1. Elsevier Science Publishing Company, Inc., New York.

Wergeland H. I. & Jakobsen R. A. (2001) A salmonid cell line (TO) for production of infectious salmon anaemia virus (ISAV). Diseases of Aquatic Organisms 44, 183-190.

Wilson M., Hsu E., Marcus A., Courtet M., Du Pasquier L. & Steinberg C. (1982) What limits affinity maturation of antibodies in Xenopus—the rate of somatic mutation or the ability to select mutants? European Molecular Biology Organization Journal 11, 4337-4347.

Wilson M. R. & Warr G. W. (1992) Fish immunoglobulins and the genes that encode them. Annual Review of Fish Diseases 201-221.

1. A method for detecting the presence or absence of antibodies to infectious salmon anaemia virus (ISAV), the method comprising the steps of:
(a) providing an ISAV or an ISAV antigen; and
(b) contacting the ISAV or ISAV antigen from step (a) with an antibody-containing sample from fish, for a time and under conditions sufficient for the antibody to form a complex with the ISAV or ISAV antigen;
wherein specific binding between the antibody and the ISAV or ISAV antigen indicates that the antibody is specific to ISAV.

2. The method according to claim 1 for testing whether a fish is or has been exposed to ISAV, wherein the antibody-containing sample is from the fish being tested, and wherein specific binding between the antibody and the ISAV or ISAV antigen indicates that the fish is or has been exposed to ISAV.

3. The method according to claim 2 wherein the fish has not been vaccinated, and wherein specific binding between the antibody and the ISAV or ISAV antigen indicates that the fish has been or is infected with ISAV.

4. The method according to claim 1, further comprising the step of:
(c) detecting the complex between the antibody and the ISAV or ISAV antigen.

5. The method according to claim 1 wherein the ISAV or ISAV antigen is immobilized.

6. The method according to claim 1 wherein specific binding between the antibody and the ISAV or ISAV antigen is detected by a labeled anti-fish antibody which binds to the complex between the antibody and the ISAV or ISAV antigen.

7. The method according to claim 1 wherein specific binding between the antibody and the ISAV or ISAV antigen is detected by contacting an immobilized anti-fish antibody with the complex between the antibody and the ISAV or ISAV antigen, wherein the ISAV or ISAV antigen is labeled.

8. The method according to claim 1 wherein the antibody-containing sample from fish has been heat-treated prior to contacting the ISAV or ISAV antigen.

9. The method according to claim 1 wherein the antibody-containing sample from fish is a fish extract.

10. The method according to claim 1 wherein the antibody-containing sample from fish is fish serum.

11. A kit for detecting the presence or absence of antibodies to ISAV in an antibody-containing sample from a fish, the kit comprising:
(a) an ISAV antigen; and
(b) means for detecting a complex between the ISAV antigen and the antibody from the fish.

12. The kit according to claim 11 wherein the ISAV antigen is immobilized.

13. The kit according to claim 12 wherein the means for detecting the complex is a labeled anti-fish antibody.

14. The kit according to claim 11 wherein the means for detecting the complex is an immobilized anti-fish antibody, and wherein the ISAV antigen is labelled.

15. A method for testing whether a fish is or has been exposed to ISAV, the method comprising the steps of:
(a) providing an ISAV or an ISAV antigen; and
(b) contacting the ISAV or ISAV antigen from step (a) with an antibody-containing sample from the fish being tested, for a time and under conditions sufficient for the antibody to form a complex with the ISAV or ISAV antigen;
(c) detecting the complex between the antibody and the ISAV or ISAV antigen;
wherein specific binding between the antibody and the ISAV or ISAV antigen indicates that the fish is or has been exposed to ISAV.

16. The method according to claim 15 wherein the ISAV or ISAV antigen is immobilized on a solid support and wherein the complex between the antibody and the ISAV or ISAV antigen is detected by detecting the antibody bound to the solid support.

17. The method according to claim 15 wherein the complex between the antibody and the ISAV or ISAV antigen is detected by contacting a labeled anti-fish antibody with the complex and detecting the labeled anti-fish antibody bound to the complex.

18. The method according to claim 15 wherein the ISAV or ISAV antigen is labeled and wherein the complex between the antibody and the ISAV or ISAV antigen is detected by contacting the complex with an anti-fish antibody immobilized on a solid support and detecting the labeled ISAV or ISAV antigen bound to the solid support.

19. The method according to claim 15 wherein the antibody-containing sample from fish has been heat-treated prior to contacting the ISAV or ISAV antigen.

20. The method according to claim 15 wherein the antibody-containing sample from fish is fish serum.
